(19) United States
(12) Patent Application Publication
(10) Pub. No.: US 2013/0096474 A1
(43) Pub. Date: Apr. 18, 2013

(54) Protective Sleeve for a Cast

(71) Applicant: Jeanie Sager, San Diego, CA (US)

(72) Inventor: Jeanie Sager, San Diego, CA (US)

(21) Appl. No.: 13/440,153

(22) Filed: Apr. 5, 2012

Related U.S. Application Data
(60) Provisional application No. 61/472,142, filed on Apr. 5, 2011.

(51) Int. Cl. A61F 5/01

(52) U.S. Cl. CPC A61F 5/01
USPC 602/3

(57) ABSTRACT

The present invention provides an improved protective sleeve comprising a character decoration for a cast worn by an individual, comprising: (a) a tubular sleeve with an opening at each end made of a stretch, fabric material for covering a cast; (b) a three-dimensional character head securely sewn to the tubular sleeve using a zigzag stitch; and (c) at least two three-dimensional character appendages securely sewn to the tubular sleeve using a zigzag stitch such that the character decoration looks similar to a stuff animal, plush toy or doll. The individual is preferably a frightened child.

### PRIORITY INFORMATION

This application claims priority from U.S. Provisional Application No. 61/472,142, filed on Apr. 5, 2011, the contents of which are hereby incorporated by reference.

### FIELD OF THE INVENTION

The present invention relates generally to a protective sleeve comprising a character decoration for a cast which makes the cast look similar to a stuffed animal, plush toy or doll while also protecting the cast from damage and/or soiling during extended wear.

### BACKGROUND OF THE INVENTION

An orthopedic cast, body cast or surgical cast is a shell, frequently made from plaster, encasing a limb to immobilize a broken bone (or bones) until healing is complete. A cast serves the same basic function as a splint, however a splint does not cover the entire limb and splints can be removed for bathing. Once casts are applied to a limb, they must remain on the limb for an extended period of time depending on the bone broken and the severity of the break. The extended time for which a cast must be worn often results in the cast becoming dirty and/or damaged. Casts are bulky and cumbersome and must be kept dry, especially when made of plaster.

Modernly, casts may be made of bandages containing synthetic materials such as knitted fiberglass. One advantage of fiberglass is that it is lighter and dries faster than plaster. Although knitted fiberglass is available in several colors and is used to create an overall brighter appearance to the cast itself, knitted fiberglass has a texture that may snag on clothing and cause abrasion to skin.

Broken bones, or fractures, are a common childhood hazard. In fact, fractures are the fourth most common injury among children under six and pediatricians are reporting a rise in the number of broken bones in children. The Mayo Clinic in Rochester, Minn., reports that over the last 30 years the number of forearm fractures in Rochester, Minn. alone has risen more than 32 percent in boys and 56 percent in girls. In most cases, when a child breaks a bone a cast will be required while the bone heals. Depending on the age of the child and the type of fracture, a cast may be on for as little as 4 weeks or as long as 10 weeks. Although some children might find a cast cool once it's applied and been on for a while, getting a cast can be a frightening experience, especially for a small child.

Frightened children find comfort in stuffed animals, plush toys and dolls. Psychologists have long used stuffed animals in therapy to help with childhood issues, such as hyperactivity, temper tantrums, staying quiet and relaxed and nightmares. Stuffed animals are also used in play therapy where a child's natural means of expression through play is used as a therapeutic method to assist the child in dealing with emotional stress or trauma. Stuffed animals, plush toys and dolls make excellent companions for children to talk to and express themselves through role play. They may even be an outlet for emotions and feelings children are not yet able to express through words. In basic terms, children find comfort and security in stuffed animals, plush toys and dolls.

Covers for casts are known in the art. Most commonly, cast covers are used to prevent the cast from getting wet during bathing. Cast covers may also be applied to allow for the individualized decoration of the cast by the person wearing it. Few, if any, cast covers with stuffed characters incorporated into the cast cover exist in the prior art. The following represent examples of cast covers and stuffed characters in the prior art.

U.S. Design Pat. No. D395,087 to Devries shows an ornamental design for a medical cast cover.

U.S. Pat. No. 4,510,928 to Ackley describes a combination sling and therapy device, open at one end and closed at the other, the closed end having a puppet-like characterization in which protruding movable lip portions are positioned to receive the thumb and fingers of the user, and at least for encouraging periodic manipulation of the thumb and fingers.

U.S. Pat. No. 4,927,025 to Thompson et al. describes a kit for maintaining and enhancing the appearance of a plaster cast including a fabric sleeve which can be used to cover a soiled cast, a waterproof cover for use when bathing, a pen set for marking on the cast and a plurality of decorative articles that may be applied to the cast.

U.S. Pat. No. 5,000,171 to Hofer describes a puppet toy having a principal portion forming the body of a rabbit, with a head and tail secured to the body, the doll primarily adapted to be disposed about a plaster cast for immobilizing a fractured arm or leg of a child by two ends with mutual engagement means that form a tubular envelope when the ends are secured to each other.

U.S. Pat. No. 6,047,403 to Juozaitis describes a decorative cast cover for a full leg cast, comprising an elongated, generally cylindrical sleeve.

U.S. Pat. No. 8,043,240 to Piatek describes a protective cover for a cast made with a resilient core for use by individuals wishing to participate in an activity while wearing a cast.

U.S. Pat. No. 8,062,087 to Davis et al. describes a glove-doll entertainment system wherein the appendages of the doll directly and removeably attach to the exterior of the glove's finger receiving cavities, and further wherein the doll is three dimensional and physically distinct from the glove.

United States Patent Publication No. US2010/0292620 by Lattimore describes a line of decorative fabric sheathes which are easily applied over any hand, wrist, arm, foot or leg cast, providing the wearer a dramatic means of enhancing the appearance of the cast and cleverly hiding it from view.

United States Patent Publication No. US2010/026057 by McClarnan describes a kit and methods for decorating a cast comprising instructions, adhesive, an adhesive applicator, a tray and at least one colored wrapping tissue paper with one or more decorative patterns.

United States Patent Publication No. US2011/0282254 by Gadlage describes a protective cover for a cast to decoratively conceal the cast and inhibit damage to clothing and the like commonly caused by the cast.

In light of the above, it is an object of the present invention to provide the desired features described herein as well as additional advantages of providing an improved protective sleeve comprising a character decoration for a cast that makes the cast look similar to a stuffed animal, plush toy or doll, the character decoration directed to the appeal of children wearing a cast and the character decoration further being inexpensive to manufacture. Another object of the present invention is to provide a means of protecting a cast from damage during extended wear as well as to protect the cast from becoming soiled during extended wear. Another object of the present invention is to provide a means of comfort and emotional support for a frightened child wearing a cast such that the character decoration aids the child in overcoming the fear and pain associated with wearing a cast.

### SUMMARY OF THE INVENTION

The present invention provides an improved protective sleeve comprising a character decoration for a cast worn by an individual, comprising: (a) a tubular sleeve with an opening at each end made of a stretch, fabric material for covering a cast; (b) a three-dimensional character head securely sewn to the tubular sleeve using a zigzag stitch; and (c) at least two three-dimensional character appendages securely sewn to the tubular sleeve using a zigzag stitch, such that the character decoration looks similar to a stuff animal, plush toy or doll. The individual is preferably a frightened child.

In a first aspect, the present invention provides an improved protective sleeve for a cast wherein the stretch, fabric material of the tubular sleeve is made of a polyurethane elastic textile having stretch and recovery properties. The tubular sleeve may further be infused with antifungal and antimicrobial compounds to prevent the growth of fungus and bacteria and reduce the likelihood of the cover developing an odor from prolonged use. The tubular sleeve, or elements within the tubular sleeve material, may further be impregnated with scented material(s) to provide a fresh or pleasant odor or seasonal scent to go with certain seasonal elements, i.e. flowers, on the sleeve. Further still, the tubular sleeve may contain padded inserts on the interior surface of the tubular sleeve to provide additional protection to the cast and/or protect objects and people in the environment external to the cast should the cast come into contact with said external objects and/or people.

In a second aspect, the present invention provides an improved protective sleeve for a cast wherein the tubular sleeve is a writing surface which may be used to provide personal, funny and/or inspiration messages to the user. The protective sleeve may further include writing instruments and/or a writing pad incorporated into the tubular sleeve.

In a third aspect, the present invention provides an improved protective sleeve for a cast comprising a character decoration wherein the character decoration is washable. The character decoration may further be non-flammable. Further still, the character decoration may be constructed of non-toxic materials and/or materials that are biodegradable.

In a fourth aspect, the present invention provides a method of using an improved protective sleeve comprising a character decoration for a cast worn by an individual, comprising: (a) locating the top opening of the tubular sleeve; (b) sufficiently stretching the top opening to allow for insertion of a limb covered by the cast; (c) inserting the limb into the top opening; and (d) pulling the tubular sleeve securely over the top of the cast.

In a fifth aspect, the present invention provides a method for using an improved protective sleeve comprising a character decoration worn by an individual in the absence of a cast, comprising: (a) locating the top opening of the tubular sleeve; (b) sufficiently stretching the top opening to allow for insertion of a limb; (c) inserting the limb into said opening; and (d) pulling the tubular sleeve securely over said limb. The covered limb may further be a wounded limb. The tubular sleeve may be comprised of a material which is breathable and provides a venting function to the injured limb and/or area. Further still, the wounded limb may contain surgical stitches. And further still, the wounded limb may contain a soft bandage. Lastly, the improved character decoration for a cast may be used in the absence of a cast for the entertainment of a child.

The invention can be better visualized by turning now to the following drawings wherein like elements are referenced by like numerals.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a side perspective view of a cast decorated to look like a stuffed animal and/or doll.

FIG. 2 is a front perspective view of a cast decorated to look like a stuffed animal and/or doll.

FIG. 3 is a front perspective view of a preferred embodiment of the present invention, i.e. a cast decorated to look like stuffed monkey.

FIG. 4 is a side perspective view of the preferred embodiment of the present invention as represented by FIG. 3, i.e. the side view of a cast decorated to look like a stuffed monkey.

FIG. 5 is an underneath perspective view of the preferred embodiment of the present invention as represented by FIG. 3, i.e. the underneath view of a cast decorated to look like a stuffed monkey.

FIG. 6 is a side perspective view of a cast decorated to look like a stuffed animal and/or doll.

### DETAILED DESCRIPTION OF THE INVENTION

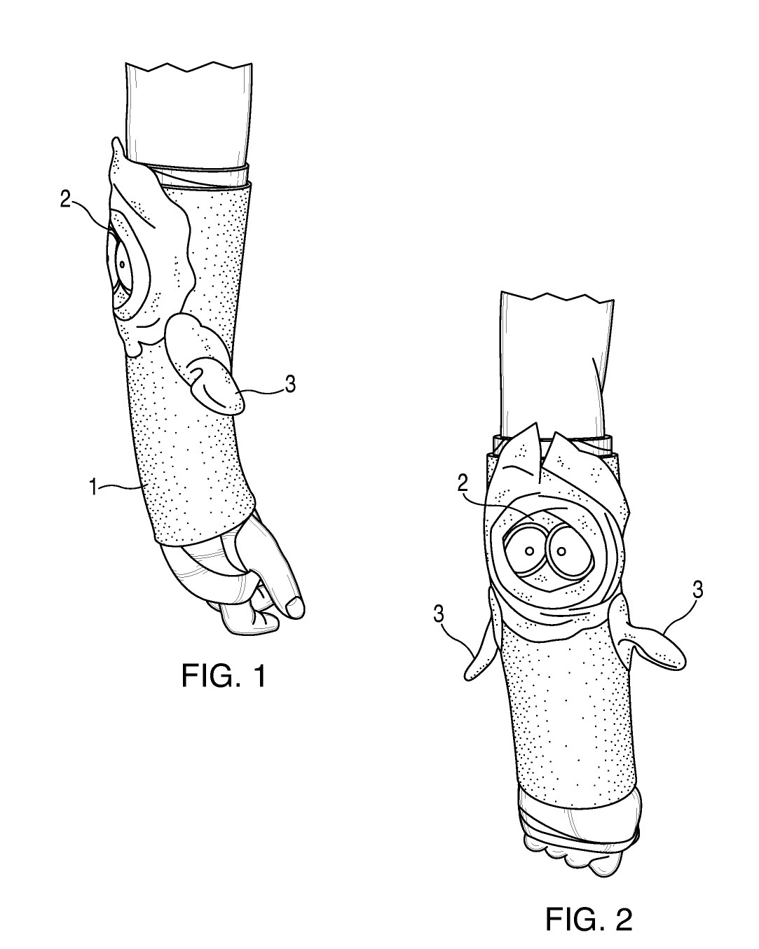

A hard cast 1 on the right arm of a child decorated to look like a stuffed animal and/or doll is illustrated in a side perspective view in FIG. 1. The face 2 of the stuffed animal and/or doll may be adhered to the upper section of the cast 1. The appendages 3 of the stuffed animal and/or doll may be adhered to the mid-section of the cast 1.

A hard cast 1 on the right arm of a child decorated to look like a stuffed animal and/or doll is illustrated in a front perspective view in FIG. 2. The face 2 of the stuffed animal and/or doll may be adhered to the upper section of the cast 1. The appendages 3 of the stuffed animal and/or doll may be adhered to the mid-section of the cast 1.

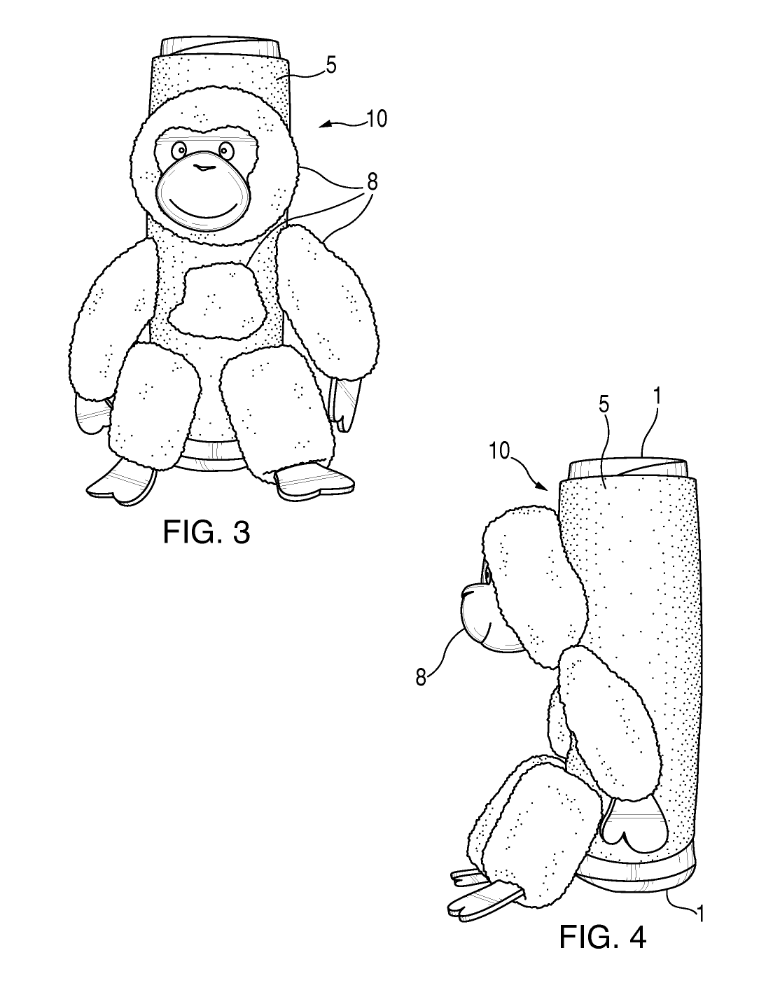

A hard cast 1 is decorated to look like a monkey in a front perspective view in FIG. 3. The main body 5 of the tubular sleeve 10 is pulled over the hard cast 1. Character body parts 8 are sewn onto the main body 5 of the tubular sleeve 10.

A hard cast 1 is decorated to look like a monkey in a side perspective view in FIG. 4. The main body 5 of the tubular sleeve 10 is pulled over the hard cast 1. Character body parts 8 are sewn onto the main body 5 of the tubular sleeve 10.

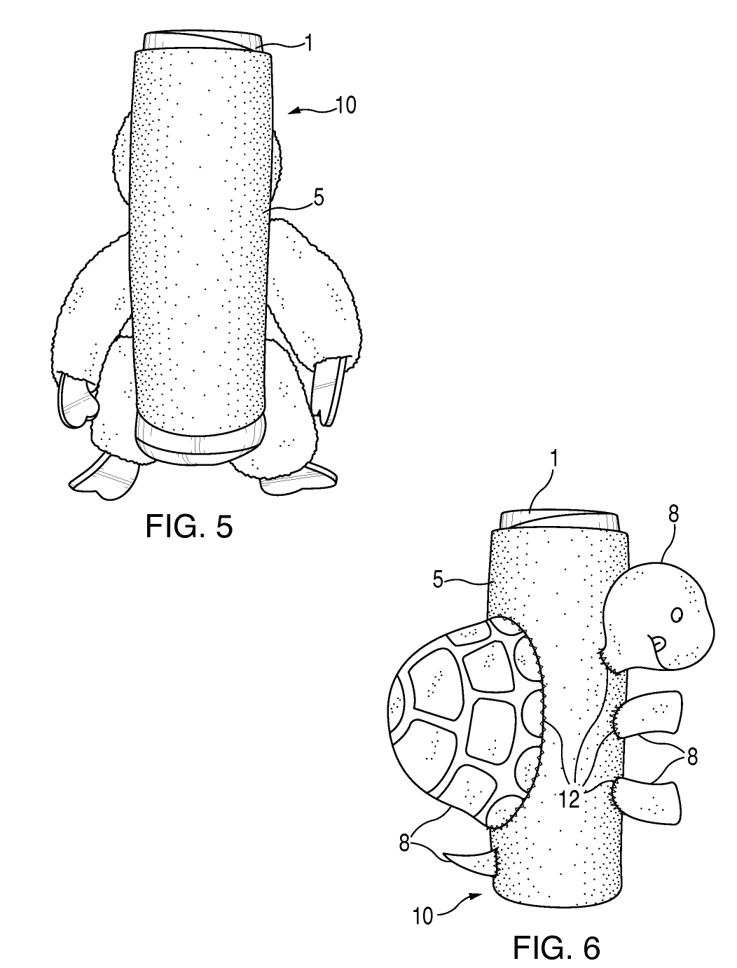

A hard cast 1 is decorated to look like a monkey in a rear perspective view in FIG. 5. The main body 5 of the tubular sleeve 10 is pulled over the hard cast 1. Main body 5 may have a reinforced panel 3 for the insertion of padded inserts to provide additional protection for the hard cast 1.

A hard cast 1 is decorated to look like a turtle in a side view perspective in FIG. 6. The main body 5 of the tubular sleeve 10 is pulled over the hard cast 1. Character body parts 8 are sewn onto the main body 5 of the tubular sleeve 10 at position 12 using a zigzag stitch. The zigzag stitching integrates the character body parts 8 into the tubular sleeve 10 such that it gives the appearance of uniformity wherein the character body parts 8 appear to be emanating from the hard cast 1 itself.

One embodiment of the present invention provides an improved protective sleeve comprising a character decoration for a cast wherein the character decoration is securely adhered to a tubular sleeve with openings at both ends.

In another embodiment of the present invention, the character decoration for a cast may be securely adhered to the tubular sleeve by sewing. Preferably the character decoration is integrated into the tubular sleeve by zigzag stitching.

In another embodiment of the present invention, the tubular sleeve may serve as a writing surface. In an alternative embodiment, a writing pad is incorporated into the tubular sleeve.

In another embodiment of the present invention, the character decoration for a cast may be securely adhered directly onto the cast by glue.

In another embodiment of the present invention, the tubular sleeve may be made from a resilient, lightweight material.

In another embodiment of the present invention, the resilient, lightweight material may be a polyurethane elastic textile having stretch and recovery properties. In a preferred embodiment, the elastic textile is SPANDEX- or LYCRA-type elastic fabric.

In another embodiment of the present invention, the tubular sleeve may contain padded inserts on the interior surface of the tubular sleeve to provide additional protection to the cast.

In another embodiment of the present invention, the padded inserts on the interior of the tubular sleeve are sufficiently placed to protect objects and/or people in the environment external to the cast should the character decoration come into contact with such objects and/or people.

In another embodiment of the present invention, the tubular sleeve may be infused with antifungal and antimicrobial compounds to prevent the growth of fungus and bacteria and to reduce the likelihood that the sleeve will develop an odor from prolonged wear.

In another embodiment of the present invention, the tubular sleeve, or elements within the tubular sleeve material, may further be impregnated with scented material(s) to provide a fresh or pleasant odor or seasonal scent to go with certain seasonal elements, i.e. flowers, on the sleeve.

In another embodiment of the present invention, the character decoration may be washable.

In another embodiment of the present invention, the character decoration may be non-flammable.

In another embodiment of the present invention, the character decoration is constructed of non-toxic materials and/or biodegradable materials.

In another embodiment of the present invention, the character decoration may include, but is not limited to, a stuffed character head, stuffed arms, stuffed body, stuffed legs, an abstract face, an animal, an object, felt attachments that are not stuffed, flowers, various shapes and patterns which may improve the appearance of a cast and add a three-dimensional quality to the cast.

In another embodiment of the present invention, three-dimensional aspects of the character decoration may be constructed of, but are not limited to, nylon, polyester, plastic, rubber, cardboard, polyurethane foam, lightweight nylon, spun polyester, plastic coated paper and lightweight stretch nylon.

In another embodiment of the present invention, the character decoration may include, but is not limited to, decorations directly printed on the tubular sleeve such as names, words, character images, hearts, smiley faces, and various images and patterns.

In another embodiment of the present invention, a method is provided for the use of the improved character decoration to provide a protective sleeve for said cast and enhance the appearance of said cast worn by an individual, comprising locating the top opening of the tubular sleeve, sufficiently stretching the opening to allow for insertion of a limb covered by a cast, inserting the limb into the opening and pulling the tubular sleeve securely over the top of a cast.

In another embodiment of the present invention, a method is provided for use of the improved protective sleeve in the absence of a cast to cover a healing wound, scar, surgical stitches or a soft bandage. Preferably the tubular sleeve of the character decoration is made of a breathable material.

In another embodiment of the present invention, a method is provided for use of the improved protective sleeve in the absence of a cast as a form of costume arm or leg covering to provide entertainment to a child.

It will be appreciated that details of the foregoing embodiments, given for purposes of illustration, are not to be construed as limiting the scope of this invention. Although several embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

What is claimed:

1. An improved protective sleeve comprising a character decoration for a cast worn by an individual, comprising:
(a) a tubular sleeve with an opening at each end made of a stretch, fabric material for covering the cast;
(b) a three-dimensional character head securely sewn to the tubular sleeve using a zigzag stitch; and
(c) at least two three-dimensional character appendages securely sewn to the tubular sleeve using a zigzag stitch such that the character decoration looks similar to a stuff animal, plush toy or doll.

2. The improved protective sleeve of claim 1, wherein the individual is a frightened or injured child.

3. The improved protective sleeve of claim 1, wherein the tubular sleeve includes a writing surface incorporated into the tubular sleeve.

4. The improved protective sleeve of claim 1, wherein the stretch, fabric material of the tubular sleeve is made of SPANDEX- or LYCRA-type elastic fabric.

5. The improved protective sleeve of claim 1, wherein the tubular sleeve is made of breathable, elastic material(s).

6. The improved protective sleeve of claim 1, wherein the tubular sleeve is infused with antifungal and antimicrobial compounds to prevent the growth of fungus and bacteria and reduce the likelihood of the cover developing an odor from prolonged use.

7. The protective sleeve decoration of claim 1, wherein the tubular sleeve is impregnated with scented material(s) to provide a fresh or pleasant odor or seasonal scent to coincide with certain seasonal elements on the sleeve.

8. The improved protective sleeve of claim 1, wherein the tubular covering further contains padded inserts on the interior surface of the tubular sleeve to provide additional protection to the cast.

9. The improved protective sleeve of claim 8, wherein the padded inserts on the interior surface of the tubular sleeve are sufficiently placed to protect objects and/or people in the environment external to the cast should the character decoration come into contact with such objects and/or people.

10. The improved protective sleeve of claim 1, wherein the character decoration is washable.

11. The improved protective sleeve of claim 1, wherein the character decoration is non-flammable.

12. The improved protective sleeve of claim 1, wherein the character decoration is constructed entirely of non-toxic materials.

13. The improved protective sleeve of claim 1, wherein the character decoration is constructed entirely of biodegradable materials.

14. A method of using an improved protective sleeve comprising a character decoration for a cast worn by an individual, comprising:
(a) locating the top opening of the tubular sleeve;
(b) stretching the top opening to allow for insertion of a limb covered by a cast;
(c) inserting the limb into the top opening; and
(d) pulling the tubular sleeve securely over the top of a cast.

15. A method of using an improved protective sleeve comprising a character decoration worn by an individual in the absence of a cast, comprising:
(a) locating the top opening of the tubular sleeve;
(b) sufficiently stretching the top opening to allow for insertion of a limb;
(c) inserting the limb into said opening; and
(d) pulling the tubular sleeve securely over said limb.

16. The method of claim 15, wherein the limb covered by the character decoration is a wounded limb.

17. The method of claim 16, wherein the tubular sleeve provides ventilation to the wounded limb.

18. The method of claim 16, wherein the wounded limb further contains surgical stitches.

19. The method of claim 16, wherein the wounded limb further contains a soft bandage.

20. The method of claim 15, wherein the use of the character decoration is for the purpose of entertaining a child.
